(19) United States
(12) Patent Application Publication
(10) Pub. No.: US 2005/0165096 A1
(43) Pub. Date: Jul. 28, 2005

(54) Pain management system

(76) Inventor: Doohi Lee, Plano, TX (US)

Correspondence Address: Doohi Lee, 5508 Ash Creek Lane, Plano, TX, 75093, US

(21) Appl. No.: 10/763,591

(22) Filed: Jan. 24, 2004

(51) Int. Cl. A61K 31/24; A61H 5/00

(52) U.S. Cl. 514/534; 600/437; 601/1

(57) ABSTRACT

A method and system is disclosed for treating pain in a body area. After obtaining at least one internal image of the body area using an ultrasound imaging tool, one or more clinically observable factors are determined from the cross section related to the pain in the first body area to determine a treatment area. The treatment area is verified by palpation while viewing the internal image through the ultrasound imaging tool. If the pain in the body area is deemed to be treatable based on the determined factors, the treatment area is prepared for treatment. The pain is treated through the prepared treatment area with assistance from the ultrasound imaging tool; and the treatment area is massaged afterward.

### BACKGROUND

The present disclosure relates generally to medical systems, and more particularly to medical procedures and systems for pain management. Still more particularly, the present disclosure relates to a system and method that uses ultrasound techniques to locate treatment areas, whereupon anesthetic and other therapeutic treatments are applied to manage bodily pain.

Pain management is a rather broad subject, spanning such subjects as orthopedics, physical therapy, anesthesiology, alternative medicine etc. The use of a combination of anesthetic and treatment drug for the management of various conditions of acute and chronic pain has been a proven technique for many years. Chronic pain includes arthritis, cancer etc., and the complaints thereof include headache, lower back pain etc. Acute and chronic pain may be caused by the tearing of muscles and tendons, partially- or fully-fractured bones etc. The level of pain is determined by the amount of nociceptors, or nerve endings located in the injured area. Muscles and other organs that are protected under the skin generally have fewer nerve endings than the skin, which may have more than one thousand nerve endings per square inch. Applying anesthetic and therapeutic treatments to a localized area has the dual effect, to a variety of degrees, of both relieving suffering and stimulating the healing process, and has been commonly practiced for many years. Typically, a treatment area for pain caused by an average muscle injury is approximately 4-6 cubic centimeters.

The difficulty with respect to the infusion of the aforesaid combination rests with the identification of the treatment area. Typically, treatment area is identified by a combination of visual inspection and palpation. Visual inspection is a technique whereby the physician attempts to locate a visible area for treatment by eyesight. For example, if the patient suffers a bodily injury, one or more small blood vessels may rupture, thereby first causing blood to be released subcutaneously and then resulting in discoloration as viewed from above the overlying skin surface. In that situation, the treatment area may be directly underneath the aforesaid discoloration. If no blood vessels are significantly ruptured, or if the injury causes body-cell damages that do not result in discoloration, visual inspection may not suffice. The physician may also have to examine by palpation, or the examination by physical touch, usually by fingertips. The physician, based on experience, may palpate over a locale (e.g. an arm, a leg etc.) and, based on irregularities during palpation, identify a smaller treatment area within the aforesaid locale. Identifiable irregularities include changes in size, consistency, texture, location and tenderness of the palpated body part. The treatment area may also be verified by the patient's response, which may be based on an increasing scale of stimulus caused by the physician's palpation as the physician palpates over the aforesaid locale.

Three problems arise with respect to conventional practices. One is the accuracy of identifying the treatment area through the patient's response. In one example, because a patient's response is based on pain signals as carried by nerve cells, the accuracy thereof depends on whether or not nerve cells are also damaged in the injury. In another example, a patient may complain that pain is felt in one arm, but may not be able to accurately point out exactly where the pain is coming from, particularly when the extent of the pain is preventing the patient from accurately doing so. Because the identified location for treatment may not be exactly the actual location, it is possible that treatment might require a dosage of anesthetic and drugs that would be more than a dosage if the treatment area were properly identified. Another problem is the inability to identify how much vertical depth the injury extends to and the degree of damage with respect to injury without cutting through the skin and examining the extent of the injury. In one example, the physician may accurately identify the location of the skin surface over which the treatment area may be. However, the physician may never know exactly how deep and how serious the injury is without using imaging technologies or cutting through the skin surface to visually diagnose the seriousness of the injury. Finally, if the injured area is not accurately and properly identified, time of treatment, type of treatment, and recovery may also be prolonged. It is well known that the dosage of anesthetic is related to the occurrence, prevalence and severity of certain side effects. For example, in chronic pain medications, the patient may build up resistance to certain anesthetic, thereby reducing the effectiveness given a certain amount of anesthetic dosage. Also, and as a practical ratter, the patient may feel that the injury is more serious than it actually is, thereby causing unnecessary fear and concern. The negative psychological effects may in turn affect the patient's ability to recover.

Desirable in the art of pain management are improved systems and methods that provide a more robust ability not only to locate the treatment area but also to accelerate the healing process.

### SUMMARY

In view of the foregoing, this disclosure provides a system and method for managing pain through the use of ultrasound techniques to locate injured areas, whereupon anesthetic and other therapeutic treatments are applied to manage bodily pain.

In one example, after obtaining at least one internal image of the body area using an ultrasound imaging tool, one or more clinically observable factors are determined from the cross section related to the pain in the first body area to determine a treatment area. The treatment area is verified by palpation while viewing the internal image through the ultrasound imaging tool. If the pain in the body area is deemed to be treatable based on the determined factors, the treatment area is prepared for treatment. The pain is treated through the prepared treatment area with assistance from the ultrasound imaging tool; and the treatment area is massaged afterward.

Various aspects and advantages will become apparent from the following detailed description, taken in conjunction with the accompanying drawings, illustrating the principles of the disclosure by way of examples.

### BRIEF DESCRIPTION OF THE DRAWINGS

FIG. 1A illustrates a pain management system in accordance with one example of the present disclosure.

FIG. 1B illustrates an application of the pain management system in accordance with one example of the present disclosure.

FIG. 2 presents a flow chart illustrating the operation of a training module in accordance with one example of the present disclosure.

FIG. 3 presents a flow chart illustrating the operation of a pain management module in accordance with one example of the present disclosure.

FIG. 4 presents a flow chart illustrating the diagnostic method in accordance with one example of the present disclosure.

FIG. 5A presents a flow chart illustrating the therapeutic method in accordance with one example of the present disclosure.

FIG. 5B presents a flow chart illustrating the preparatory steps in accordance with one example of the present disclosure.

FIG. 5C presents a flow chart illustrating the therapeutic steps in accordance with one example of the present disclosure.

### DESCRIPTION

The present disclosure provides an improved system and method for managing pain. Ultrasound techniques are utilized to locate treatment areas, whereupon anesthetic and other therapeutic treatments are applied to manage the bodily pain in these treatment areas.

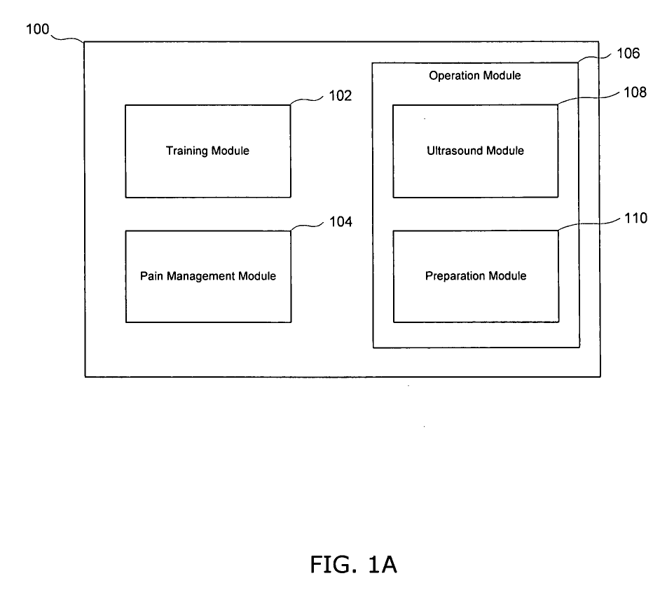

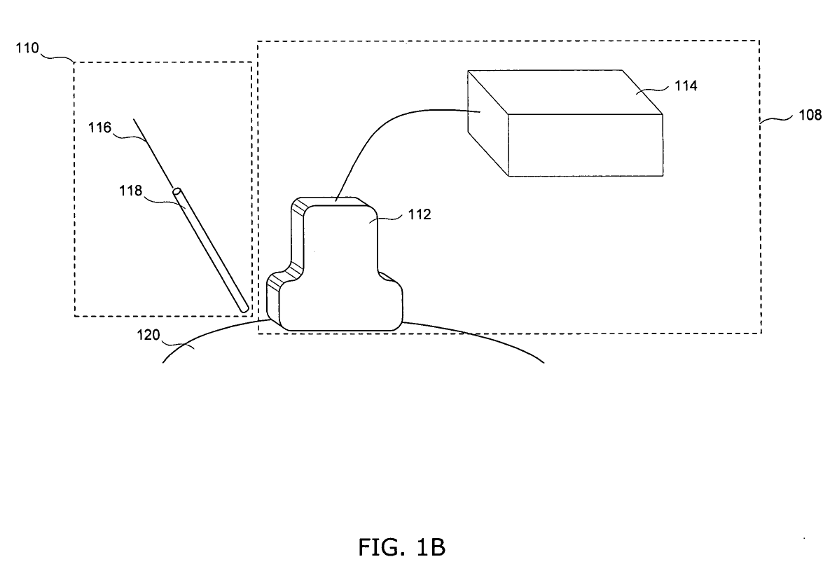

FIG. 1A illustrates a pain management system 100 in accordance with one example of the present disclosure. The pain management system 100 is a combination of software and hardware products, and may be provided to a clinic as a complete solution package that includes a training module 102, a pain management module 104, and an operation module 106. The operation module 106 may further include an ultrasound module 108 and a preparation module 110.

The training module 102 includes one or more software or manuals providing various training processes and methods to train the operators of the pain management system 100. An operator may be a medical doctor, a nurse, or any medical personnel authorized to operate the aforesaid pain management system 100.

As an example, the training module 102 may include a full set of learning materials designed to train operators who are not familiar with the pain management system 100, and a partial set of learning materials designed to train operators who have prior experience with the pain management system 100 but whose skills may require retraining due to upgrades and/or updates thereto. These materials may include learner manuals, demonstration videos, a step-by-step instruction set and other training tools or computer software that train an operator to operate the pain management system 100.

In one example, the learner manuals may include the features, philosophy, and the techniques that give the operator a basic theoretical conception of the pain management system 100. The demonstration video or software program may contain video clips of physicians demonstrating how to use the pain management system 100. In other words, these video clips are practical examples embodying the theoretical conceptions as presented in the aforesaid learner manuals. A step-by-step instruction set may include a step-by-step manual that teaches the operator how to operate the pain management system 100. In one example, the step-by-step manual may further include a list of learning outcome statements, learning modules, and quizzes designed to test the acquired knowledge of the operator. Other training tools may include testing kits, retraining manuals for operators with prior experience with the pain management system 100, and other materials deemed relevant in training the operator how to operate the pain management system 100 and its corresponding modules.

The pain management module 104 includes one or more software programs or other manuals providing functional processes and methods to guide the operator during the operation of the pain management system 100. Some of these materials may be similar to those included in the training module 102, except that operator using the materials in the pain management module 104 is expected to have completed the training process as defined in the training module 102.

For example, the pain management module 104 may include operator manuals, quick reference cards, step-by-step flowcharts on how to proceed after a certain step, emergency manuals dealing with emergencies and mistakes, safety guides, and other materials deemed relevant in the actual operation of the pain management system 100. Operator manuals may include detailed, step-by-step guide to operating the pain management system 100. Quick reference cards may include a brief list of important steps that the operator must pay careful attention to. Step-by-step flowcharts may include an exhaustive flow chart that allows the operator to look up what is to be performed after a certain step, just in case the operator is unsure of what the next step may be. Emergency manuals may include steps detailing how to deal with particular emergencies. For example, the power supply may be cut off from the ultrasound probe during a power outage. The operator must then proceed to the emergency manual to look up what next steps must be accomplished to ensure the safety of the patient and the proper calibration of the ultrasound probe. Safety guides may include summaries of what must be actively considered and followed during the operation of the pain management system 100. For example, the safety guide may include steps detailing how to dispose different disposable items. Certain items may be disposed immediately (e.g. dressing materials, sterilization materials etc.), while other items may have to be protected before disposal (e.g. needles, blades etc.).

The hardware part of the pain management system 100 includes the ultrasound module 108, which has all the equipments necessary to operate an ultrasound probe. For example, the ultrasound module 108 may include an ultrasound probe, a power supply/transformer that provides electrical power to the said probe, a power cable connectable between the said probe and the said power supply/transformer, a digital computer for analyzing and presenting the data captured from the said probe, and a cable connectable between the said probe and the said digital computer. The ultrasound probe may be embodied by different designs. For example, the ultrasound probe may have a linear or a curved detection array. The digital computer may include one or more of the following items: a processing unit, input devices, a video output, printing devices etc.

The ultrasound module 108 may also include technical manuals for operating a plurality of the aforesaid equipments. As an example, these technical manuals may include information to power-cycle the ultrasound probe, to replace defunct batteries, and to reset calibration parameters etc. It is understood by those skilled in the art that these technical manuals may include other information guiding the operator to properly perform certain technical functions. It is also understood by those skilled in the art that the operator may opt to use other equivalent components that are not included in the pain management system, and that the module 108 may provide manuals indicating how to connect one or more equipments of the ultrasound module 108 to other external units. For example, the operator may opt to use an external digital computer in lieu of the digital computer that may be provided in the pain management system 100. The operator may do so by reading the corresponding section of a manual providing step-by-step instruction as to how to connect the said external digital computer to the pain management system 100.

The preparation module 110 may include a lancing equipment set, an anesthetic set, a medical drugs set, a preparation set and other items that may be used in preparation for therapy and during therapy. The lancing equipment set may include a lancing needle and a needle guide through which the lancing needle may be directed. The anesthetic set may include one or more ready-made cocktail dosages of anesthetic. Various cocktail dosages with a variety of concentrations are designed to allow the operator to select the correct cocktail dosage as determined in diagnosis. A cocktail dosage of localized anesthetic may include local anesthetics such as lidocaine, etidocaine, bupivacaine and others. These local anesthetics typically produce pain relief by blocking signals at the nerve endings. The medical drugs set may include one or more ready-made cocktail dosages of medical drugs. Medical drugs may include antibiotic and other chemicals that may provide one or more of the following functions: stimulation of the healing process, reduction of blood clotting etc. The preparation set may include surgical drapes, sterilization kits, clinical gloves, syringes and other items that may be used during the operation of the pain management system 100.

While the above illustration of the pain management system 100 may include a variety of equipments and items, it is understood by those skilled in the art that other equipments and items may be added, and that some of the discussed equipments and items may be omitted from the packaged pain management system 100 without significant deviation to the principles and the spirit of the disclosure.

FIG. 1B presents an application of the operation module 106 of the pain management system 100 in accordance with one example of the present disclosure. With reference to FIGS. 1A and 1B, the application set forth in FIG. 1B includes equipments set forth in the discussion of the ultrasound module 108 and the preparation module 110. To simplify the illustration, not all the elements of the operation module 106 are shown. In one example, the equipments in the ultrasound module 108 include an ultrasound probe 112 and a digital computer 114, while the equipments in the preparation module 110 include an injection needle 116 and a needle guide 118. A body area 120 presents the injury locale, in which a treatment area exists and must be identified. As an example, the body area suffers a bruise, thereby causing physical damage to one or more muscles and damages to other relevant parts of the body. When the operator moves the ultrasound probe 112 around the body area 120, an ultrasound image may be displayed in the digital computer. Based on the viewable image, the operator decides where the treatment area may be. For example, the cross section of a healthy muscle may show uniformly distributed muscle fibers, while the cross section of a bruised or injured muscle may show unevenly distributed muscle fibers or even broken fibers. The operator may also decide how serious the injured muscle may be by looking at a variety of factors, including the angle of pennation, bundle length of the muscle, the uniformity of relaxed and contracted muscles and other clinically observable factors. The ultrasound image may also identify the integrity of tendons, the localization of foreign bodies, abscesses, hermatomas and other fluid collections that are instrumental in deciding how serious the injury may be.

The treatment area identified through the ultrasound image may then be verified by a series of palpation. Based on the operator's experience and feedback from the patient, the verified treatment area may then be located. If the viewable image does not indicate a possible treatment area, the operator may move to a different portion of the body area 120 until the treatment area is identified.

Once the treatment area is identified, the operator may decide whether to proceed to begin a therapy session or end the pain management session. The latter is possible for injuries that may not be treatable by the pain management system 100, or that may be better served by other medical procedures. If the operator decides to begin a therapy session, the operator may start preparing for therapy.

Preparation may include a variety of steps as illustrated in the aforesaid operating manuals in the pain management module 104. In one example, it is first determined that specific levels of anesthetic and medical drugs are required for a particular treatment area. The operator then selects the correct cocktail dosages of anesthetic and medical drugs from the preparation module 110 and applies the dosage into their corresponding injection needles. For example, for a particular injury, it may be determined that a cocktail dosage with 40 percent of 2% lidocaine (short-acting) and 60 percent of 0.25% bupivacaine (medium-acting) is appropriate. For each dosage of anesthetic and medical drugs, the injection needle 116 is inserted into the body area 120 through the needle guide 118. The angle and the depth of the insertion are determined by the ultrasound image and may be adjusted as the operator inserts the needle into the body area 120.

After injection of anesthetic and medical drugs, the operator may use the injection needle 116 as a lancing needle to stimulate the muscles, thereby jump-starting the healing process. The injection needle 116 may also be removed and replaced with a separate lancing needle that may have a different needle size. The term “lancing needle” is used interchangeably between the aforesaid separate lancing needle and the injection needle 116 that is also used as a lancing needle. The operator may also be able to distribute anesthetic and medical drugs more evenly by looking at the real-time ultrasound image and working the tip of the lancing needle. The combination of lancing and the use of ultrasound data to assist lancing is hereinafter referred to as sonolancing.

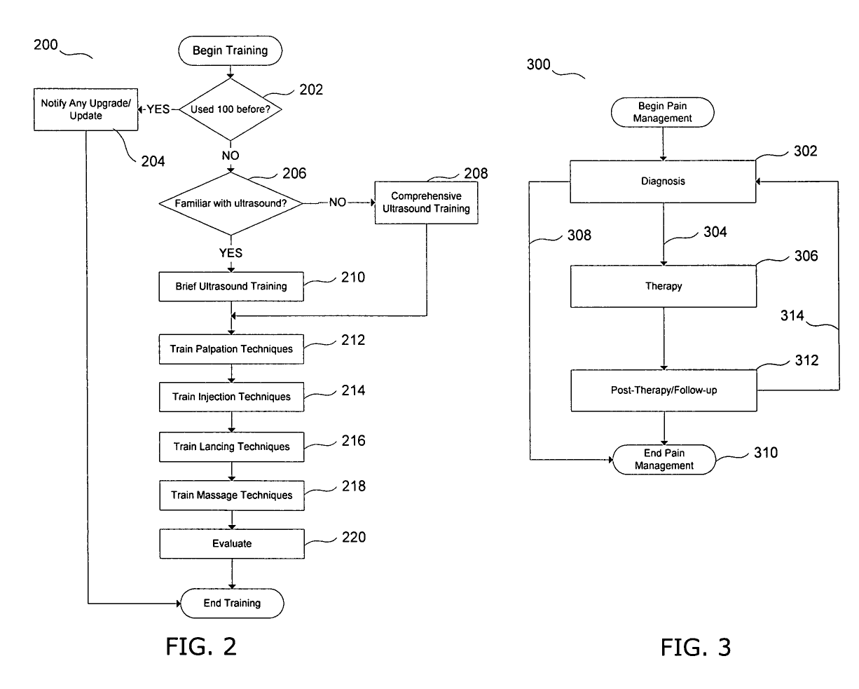

After the lancing operation is complete, the lancing needle and the needle guide may be removed, and therapy continues with a series of massaging while the ultrasound probe is still providing ultrasound images. By massaging the treatment area, medication may be spread more evenly throughout the treatment area. After the series of massaging is complete, the operator applies sterile dressing procedures and then completes the therapy session. The aforementioned steps will be discussed in further details in FIGS. 2 to 5C.

FIG. 2 presents a flow chart 200 illustrating the operation of the training module 102 in accordance with one example of the present disclosure. The training module may provide a training software program that conducts a training session. When the training session begins, it is determined, through step 202 of the flow chart 200, whether the operator has used the pain management system 100 before. If the operator has used the pain management system 100 before, the operator is presented, as depicted in step 204, with a partial set of learning materials designed to notify the operator the upgrades and/or updates since the last time the operator has used the pain management system 100. After the operator learns the upgrades and/or updates, the training session ends.

If the operator has not used the pain management system 100 before, or if the operator has used the pain management system 100 before but has since forgotten much of its functionalities, the operator may be presented with a full set of learning materials. The flow proceeds by asking, as depicted in decision box 206, whether the operator is familiar with operating an ultrasound probe. If the answer is no, the operator is presented, as depicted by step 208, with a comprehensive ultrasound training. If the answer is yes, the operator is presented with a brief ultrasound training, as depicted by step 210. After the completion of either case, the flow proceeds to step 212, where the operator is trained with palpation techniques. These techniques may include instructions on how to positively identify irregularities, how to reject irregularities that are not otherwise caused by the injury, and techniques on how to move the fingertip to best palpate skin surface on top of the treatment area without causing further damage thereto. After the palpation training is complete, the flow proceeds to step 214, where the operator is trained with injection techniques. These techniques may include instructions on how to hold the needle, how to inject fluids from cocktail dosage bottles thereto, how to use the needle guide, and how to insert the injection needle into the body. After the injection training is complete, the flow proceeds to step 216, where the operator is trained with lancing techniques. These techniques may include instructions on how to hold the lancing needle, how to lance the treatment area based on certain ultrasound imageries, and how to retract the lancing needle after lancing is complete. The flow then proceeds to step 218, where the operator is trained with massaging techniques. These techniques may include instructions on how to massage the treatment area based on certain ultrasound imageries. In particular, the instructions may include speed, pressure and directional requirements for a certain type of injuries as identified during diagnosis. The flow then proceeds to step 220, where the operator is evaluated. Based on the evaluation, the operator may successfully complete the training, or may be required to undergo part of or all of the training steps.

FIG. 3 presents a flow chart 300 illustrating the operation of the pain management module 104 in accordance with one example of the present disclosure. A pain management session is an actual session with a real patient and real equipments for the operation of the pain management system 100. The pain management session begins in a step 302, where diagnosis is performed. If the diagnosis determines that therapy is needed, the flow proceeds, through a path 304, to a step 306, where a therapy session is to begin. If the diagnosis determines that therapy is not needed, or that the injury may be best treated with other pain management techniques other than those provided by the pain management system 100, the flow proceeds, through a path 308, to a terminating step 310, where the pain management session ends.

After the therapy session ends, if a therapy session is required at all, the flow proceeds to a step 312, where a post-therapy follow-up session is to begin. Post-therapy follow-up may include a variety of diagnostic tests, sample retrieval and analysis, and interviews with the patient. The pain management system 100 may include a post-therapy follow-up “check-list” for the operator to check off all the follow-up checking points. If it is determined that further therapy is required, the flow returns, through a path 314, to the step 302, where further diagnosis is performed, and then to the step 312, where further therapy is performed. If it is determined that no further therapy is required, the flow proceeds to the step 310, where the pain management session ends. It is also possible that further post-therapy follow-ups may occur several days or weeks after the end of the pain management session. In those follow-ups, the pain management session may be restarted at the step 302 or the step 306, or at any steps within step 302 or step 306 that will be further discussed in the following paragraphs. It is also possible that the level of anesthetic and medical drugs applied may change, including the addition of steroid medications and others that may be introduced to continue the healing process.

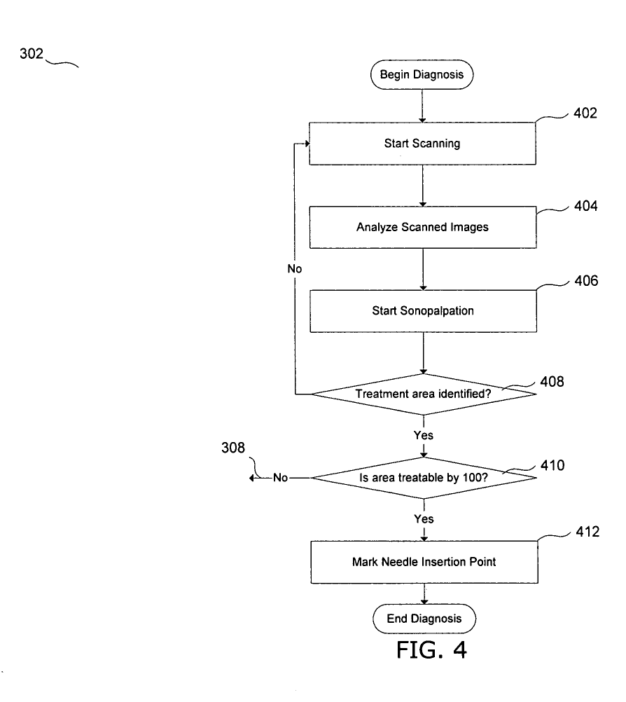

FIG. 4 presents a flow chart illustrating the diagnostic method in accordance with one example of the present disclosure. With reference to both FIGS. 3 and 4, the flow chart in FIG. 4 is essentially a detailed breakdown of the step 302. When a diagnosis session begins, the operator begins by applying a conducting gel onto the body area and then by scanning the body area with an ultrasound probe in a step 402. Scanned images are then analyzed in a step 404. If it is determined that a treatment area may be found, the operator palpates while the ultrasound probe continuously scans the body area, as depicted by a step 406. The combination of palpation and the use of ultrasound data to assist palpation is hereinafter referred to as sonopalpation. The flow then proceeds to a decision box 408, where it is determined whether the treatment area is identified. If the answer is no, the flow returns to the step 402, where scanning continues until the treatment area is found. If the answer is yes, the flow proceeds to a decision box 410, where it is determined whether the treatment area may be treatable by the pain management system 100. If it is determined that the treatment area may not be treatable by the pain management system 100, the flow proceeds, through the path 308, to the terminating step 310, where the pain management session ends. If it is determined that the treatment area may be treatable by the pain management system 100, the flow proceeds to a step 412, where the needle insertion point is marked on the skin surface. After the treatment area is marked, the flow proceeds to end the diagnosis session.

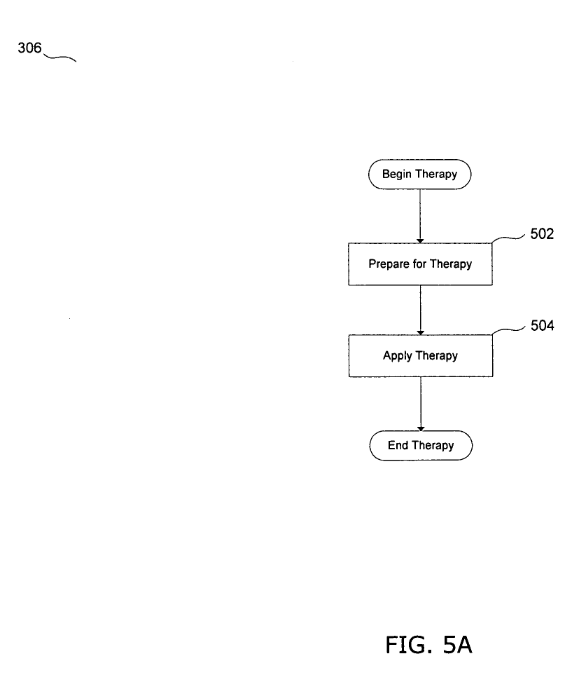

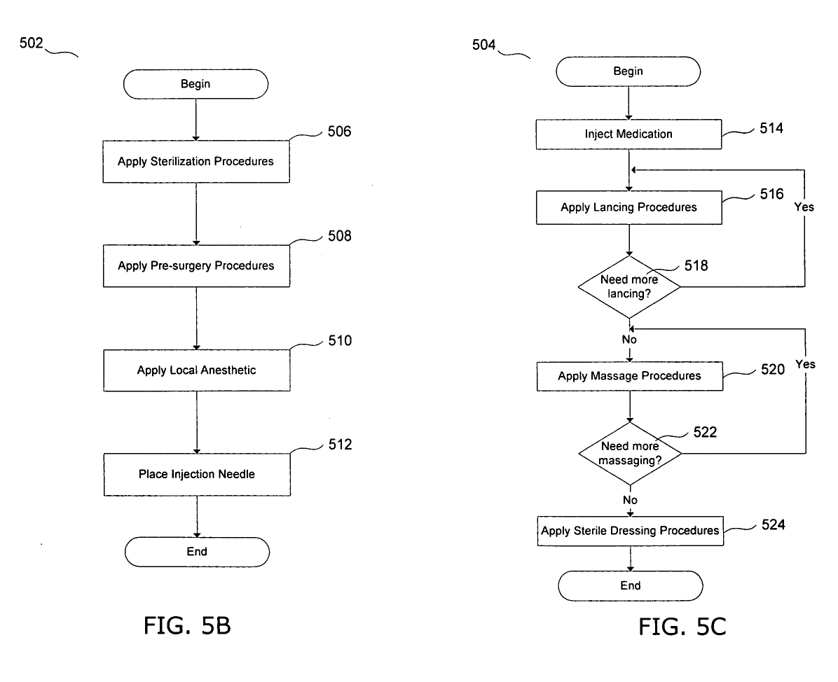

FIG. 5A presents a flow chart illustrating the therapeutic method in accordance with one example of the present disclosure. With reference to both FIGS. 3 and 5A, the flow chart in FIG. 5A is essentially a detailed breakdown of the step 306. When a therapy session begins, the operator begins by preparing for therapy, as depicted in a step 502. When the operator completes preparing for therapy, the flow proceeds to a step 504, where the operator applies the therapy to the treatment area identified in the step 302. Steps within steps 502 and 504 will be further discussed below.

FIG. 5B presents a flow chart illustrating the preparatory steps in accordance with one example of the present disclosure. With reference to both FIGS. 5A and 5B, the flow chart in FIG. 5B is essentially a detailed breakdown of the step 502. A preparation session begins in a step 506, where the operator applies sterilization procedures to the treatment area. For example, the operator may be required to sterilize the tip of the injection needle, the skin surface through which the injection needle will pierce, and other equipments and items that may be in direct contact with the lesion. After equipments and items are sterilized, the flow proceeds to a step 508, where certain pre-surgery procedures are applied. For example, injection needles with a particular friction, size and sharpness may be selected based on the area through which the needles may have to pierce and the amount of fluids that the needles may have to deliver. Other factors may also have to be considered, including tissue stiffness and compressibility, which are determined by sonopalpation. Also, a correct cocktail dosage of anesthetic and medical drugs may be chosen and transferred from their containers into their corresponding injection needles. Other pre-surgery procedures include draping, wearing of a clinical gown, the introduction of fluid collection pouches and other preparations necessary for the subsequent steps. After pre-surgery procedures are completed, the flow proceeds to a step 510, where local anesthetic is applied. With reference to both FIGS. 4 and 5B, after the local anesthetic is applied, the flow proceeds to a step 512, where the injection needle is placed in the direction towards the needle insertion point as marked in the step 412. After the injection needle is placed, the preparation session ends.

FIG. 5C presents a flow chart illustrating the therapeutic steps in accordance with one example of the present disclosure. With reference to both FIGS. 5A and 5C, the flow chart in FIG. 5C is essentially a detailed breakdown of the step 504. A therapy session begins in a step 514, where the operator injects medical drugs to the treatment area. The insertion of the injection needle is guided by a needle guide, and more importantly, by the real-time ultrasound image that shows the viability of the insertion. If it is determined that the insertion is at a wrong angle, the needle may be quickly retracted and realigned such that it may be aligned correctly. Also, the ultrasound image may show exactly how the medical drugs, in the form of fluids, are injected into the treatment area. If the medical drugs are determined to have concentrated too much in one area and too little in another, further steps, to be further discussed below, may be required to ensure that the medical drugs are spread around the treatment area evenly.

After medical drugs are injected, lancing procedures are applied in step 516. By using certain lancing procedures, the operator may be able to stimulate the muscles, thereby jump-starting the healing process. For example, the aforementioned sonolancing techniques are applied, not only to stimulate the muscles, thereby jump-starting the healing process, but also to distribute medical fluids more evenly. The flow then proceeds to a decision box 518, where it is determined whether more lancing is needed. If the answer is yes, the flow returns to the step 516, where more lancing procedures are applied. If the answer is no, the flow proceeds to step 520, where massage procedures are applied. By massaging the treatment area, medication may be spread more evenly throughout the treatment area. The flow then proceeds to a decision box 522, where it is determined whether more massaging is needed. If the answer is yes, the flow returns to the step 520, where more massaging procedures are applied. If the answer is no, the flow proceeds to a step 524, where sterile dressing procedures are applied. For example, the needle insertion area may be covered with ointment, gauze, or both, depending on the size and the occlusive requirements of the lesion caused by the needle insertion.

After sterile dressing procedures are applied, certain disposal procedures are followed. For example, all disposable items such as syringes, extra dressing materials and sterilization materials may be discarded without further procedures. The sharp ends of the injection and lancing needles may need to be secured in a protective container before disposal. For example, the protective container for injection and lancing needles must be tightly closed and labeled with a biohazard symbol, if indeed appropriate, before disposal. Unused anesthetic and medical drugs must be discarded or stored in accordance with procedural and safety guidance. After all sterile dressing procedures and disposal procedures are completed, the therapy session ends.

The above disclosure provides many different embodiments, or examples, for implementing different features of the disclosure. Specific examples of components, and processes are described to help clarify the disclosure. These are, of course, merely examples and are not intended to limit the disclosure from that described in the claims.

Although illustrative embodiments of the disclosure have been shown and described, other modifications, changes, and substitutions are intended in the foregoing disclosure. Accordingly, it is appropriate that the appended claims be construed broadly and in a manner consistent with the scope of the disclosure, as set forth in the following claims.

1. A method for treating pain in a body area, the method comprising:
obtaining at least one cross section of the body area using an ultrasound imaging tool;
determining one or more clinically observable factors from the cross section related to the pain in the body area to determine a treatment area;
verifying the treatment area by palpation while viewing the cross section through the ultrasound imaging tool;
if the pain in the body area is deemed to be treatable based on the determined factors, preparing the treatment area for treatment;
treating the pain through the prepared treatment area with assistance from the ultrasound imaging tool; and
massaging the treatment area.

2. The method of claim 1 wherein the treating the pain further includes injecting one or more anesthetic and medical drugs into the treatment area under a guidance of the ultrasound imaging tool.

3. The method of claim 2 wherein the treating the pain further includes stimulating muscles in the treatment area using a needle for accelerating a healing process of the body area.

4. The method of claim 2 wherein the anesthetic and medical drugs include a combination of lidocaine and bupivacaine.

5. The method of claim 4 wherein the anesthetic and medical drugs include a combination of about 40 percent 2% lidocaine and about 60 percent 0.25% bupivacaine.

6. The method of claim 2 wherein the injecting further includes inserting an injection needle with a predetermined insertion angle as guided by a needle guide.

7. The method of claim 1 wherein the massaging is conducted under the guidance of the ultrasound imaging tool.

8. The method of claim 1 wherein the clinically observable factors include an angle of pennation, bundle length of muscles, uniformity of relaxed and contracted muscles.

9. The method of claim 1 wherein the preparing further includes marking a needle insertion point.

10. A method for treating pain in a body area, the method comprising:
obtaining at least one internal image of the body area using an ultrasound imaging tool;
determining one or more clinically observable factors from the cross section related to the pain in the body area to determine a treatment area;
verifying the treatment area by palpation while viewing the internal image through the ultrasound imaging tool;
if the pain in the body area is deemed to be treatable based on the determined factors, preparing the treatment area for treatment;
injecting one or more anesthetic and medical drugs into the treatment area under a guidance of the ultrasound imaging tool;
stimulating muscles in the treatment area using a lancing needle for accelerating a healing process of the body area; and
massaging the treatment area.

11. The method of claim 10 wherein the anesthetic and medical drugs include a combination of lidocaine and bupivacaine.

12. The method of claim 11 wherein the anesthetic and medical drugs include a combination of about 40 percent 2% lidocaine and about 60 percent 0.25% bupivacaine.

13. The method of claim 10 wherein the injecting further includes inserting an injection needle with a predetermined insertion angle as guided by a needle guide.

14. The method of claim 10 wherein the massaging is conducted under the guidance of the ultrasound imaging tool.

15. The method of claim 10 wherein the clinically observable factors include an angle of pennation, bundle length of muscles, uniformity of relaxed and contracted muscles.

16. The method of claim 10 wherein the preparing further includes marking a needle insertion point.

17. A method for treating pain in a body area, the method comprising:
obtaining at least one internal image of the body area;
determining one or more clinically observable factors from the internal image related to the pain in the body area to determine a treatment area;
verifying the treatment area by palpation while viewing the internal image;
if the pain in the body area is deemed to be treatable based on the determined factors, preparing the treatment area for treatment;
injecting one or more anesthetic and medical drugs into the treatment area;
stimulating muscles in the treatment area using a lancing needle for accelerating a healing process of the body area; and
massaging the treatment area,
wherein the obtaining, determining, verifying, injecting, stimulating, and massaging steps are all performed under a guidance of an ultrasound imaging tool.

18. The method of claim 17 wherein the anesthetic and medical drugs include a combination of lidocaine and bupivacaine.

19. The method of claim 17 wherein the injecting further includes inserting an injection needle with a predetermined insertion angle as guided by a needle guide.

20. The method of claim 17 wherein the preparing further includes marking a needle insertion point.
